(19) United States
(12) Patent Application Publication
(10) Pub. No.: US 2010/0289006 A1
(43) Pub. Date: Nov. 18, 2010

(54) Novel blue organic compound and organic electroluminescent device using the same

(76) Inventors: Chin-Hsin Chen, Taipei (TW); Yao-Shan Wu, Taipei (TW); Meng-Huan Ho, Taipei (TW); Shih-Wen Wen, Taipei (TW)

Correspondence Address: Jackson Intellectual Property Group PLLC, 106 Starvale Lane, Shipman, VA, 22971, US

(21) Appl. No.: 11/826,039

(22) Filed: Jul. 11, 2007

(30) Foreign Application Priority Data
May 18, 2007 (TW) 096117874

(51) Int. Cl. H01L 51/54; C07C 211/54

(52) U.S. Cl. 257/40; 564/434; 564/429; 564/427; 257/E51.027

(57) ABSTRACT

Novel blue organic compound is provided. Using the blue organic compound, an organic electroluminescent device is provided, which achieved a blue emission with high efficiency, saturated color and long device lifetime. The novel blue organic compound is represented by the following general formula (1).

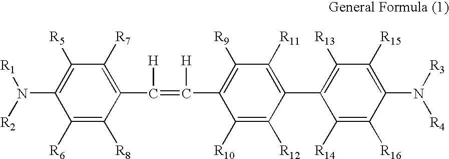

wherein R₁, R₂, R₃, and R₄ represent a substituted or unsubstituted aryl group from 6 to 20 carbon atoms, in which R₁, R₂, R₃, and R₄ may be identical with or different from each other, or R₁-R₂ and R₃-R₄ may be bridged to 5 to 7-membered carbocyclic ring. R5 to R16 represent hydrogen or a substituted or unsubstituted alkyl or aryl group from 1 to 10 carbon atoms. Besides, R₁-R₅, R₂-R₆, R₃-R₁₅, R₄-R₁₆, R₅-R₇, R₆-R₈, R₉-R₁₁, R₁₀-R₁₂, R₁₃-R₁₅ and R₁₄-R₁₆ may be bridged to a carbocyclic ring from 3 to 10 carbon atoms.

### BACKGROUND OF THE INVENTION

(a) Field of the Invention

The present invention is related to a novel blue organic compound and organic electro-luminescent device using the same, and more particularly, to a blue device of high light emission efficiency, saturated color, and long device life that is obtained from the novel blue organic compound of the present invention.

(b) Description of the Prior Art

An organic electro-luminescent device for providing many advantages including self light emission, light and thin, power-saving, and wider angle of vision (160° or greater), high responding speed, and full coloration has been taken as the most ideal flat display technology. Research of the organic electro-luminescent device may be traced back to the anthracene single chip luminescent using externally applied voltage attempted by Pope et al in 1962; however, their works received very little attention at that time due to comparatively higher working voltage (400V). Later C. W. Tang and S. A. VanSlyke (Appl. Phys. Lett. 50, 913 (1987)) developed a dual-layer organic electro-luminescent device containing electronics and electric hole transmission layers using a thermal vapor disposition method, wherein a material used in the electronic transmission layer not only functions to transmit electrons but also becomes a light emitting material and a multi-layer device structure significantly reduce a working voltage of the device; accordingly, even with a working voltage as low as less than 10V, more than 1000 cd/cm² luminance and 1% of external quantum efficiency can be achieved. Their teachings of C. W. Tang and S. A. VanSlyke can be seen in U.S. Pat. No. 4,539,507 B, U.S. Pat. No. 4,720,432 B, and U.S. Pat. No. 4,885,211 B. Furthermore, C. W. Tang, S. A. VanSlyke and C. H. Chen (Appl. Phys. Lett. 65, 3610 (1989)) proposed a concept of primary-secondary luminescent system, wherein a primary luminescent object is admixed with a secondary object of fluorescence or phosphorescence with high luminescent efficiency to promote light emitting efficiency and long service life of a device through an energy conversion mechanism between the primary and the secondary objects while achieving different colors of emission depending on an individual secondary emission material with their teachings disclosed in U.S. Pat. No. 5,151,629 B, U.S. Pat. No. 5,409,783 B, U.S. Pat. No. 5,382,477 B, JP 2-247278 A, JP 3-255190 A, JP 5-202356 A, JP 9-202878 A, and JP 9-227576 A. These two important technical developments successfully pushed the organic electro-luminescent device into the application field of full coloration flat display.

In applying the organic electro-luminescent device in the field of full coloration flat display, how to obtain an organic electro-luminescent material that provides high light emitting efficiency, saturated color and long device life becomes a crucial element. Novel blue material among others becomes a focus of research whereas it is very difficult to have a novel blue organic material that is of high efficiency and saturated color (a value of y in CIE color shade coordinates was demanded to be less than 0.15 in 1931).

In the previous studies of novel blue organic materials, a series of organic materials present by Idemitsu Kosan, Japan was most prominent. In 1996, Idemitsu Kosan present a blue secondary emission material having diamino substituted stilbene primary configuration (refer to Japanese Patent No. 8-239655 granted on Sep. 17, 1996); a device based on the blue secondary emission material achieves 10 cd/A light emitting efficiency and long device life; however, when a color of light emitted by device is measured with the 1931 CIE coordinates (x=0.17, y=0.32), value of y is found comparatively higher (more whitish) making the series of novel primary luminescent materials not suitable for application in a full coloration flat display. Later Idemitsu Kosan launched a new series of blue secondary emission material in 2003 to obtain bluer emission wave by shortening the counts of styrene among diaminos (refer to US 2003/0044640 granted on Mar. 6, 2003) ; value of y in color shade coordinates of a device based on the new series as light emitting material falls between 0.16˜0.19, with a light emitting efficiency of 4.1˜4.5 cd/A. Though having made significant improvement in color saturation for the device, both of highest occupied molecular orbitals (HOMO) and lowest unoccupied molecular orbitals (LUMO) of the new series do not fall in the primary emission due to that in the structure of the device, electronics are pushed by diamino radicals to restrict further incorporation of electronics/electric holes in the admixture.

Furthermore, in an organic compound of single amino substituted styrene configuration, similar materials have been applied as red organic materials (refer to U.S. Pat. No. 6,680,131 granted on Jan. 20, 2004) but not materials as needed in blue emission.

### SUMMARY OF THE INVENTION

The primary purpose of the present invention is to provide a novel blue organic compound summarized with an organic compound structure to correct defectives found with blue emission materials of the prior art by adding benzene rings to one end of stilbene as represented by Formula I. The structure weakens the ability of pushing electronics by diamino radicals thus to change its energy levels for the structure to better comply with energy levels of the blue primary emission. Wherein, R₁, R₂, R₃, and R₄ respectively represent substituted or un-substituted or aryl group from 6 to 20 carbon atoms while R₁, R₂, R₃, and R₄ may be identical with or different from one another, or R₁-R₂ and R₃-R₄ may be bridged to 5 to 7-membered carbocyclic ring; R₅ to R₁₆ represent hydrogen or a substituted or un-substituted alkyl or aryl group from 1 to 10 carbon atoms. Furthermore, R₁-R₅, R₂-R₆, R₃-R₁₅, R₄-R₁₆, R₅-R₇, R₆-R₈, R₉-R₁₁, R₁₀-R₁₂, R₁₃-R₁₅, and R₁₄-R₁₆ may be bridged to a saturated or unsaturated carbocyclic ring from 3 to 10 carbon atoms.

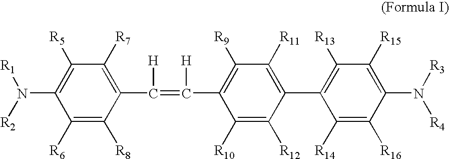

Another purpose of the present invention is to provide an installation of an organic electro-luminescent device, which achieved a blue emission with high efficiency, saturated color and long device lifetime. The organic electro-luminescent device is comprised of one or a plurality of pair of electrodes, a single layer or multiple layer structure containing an anode, a cathode, and organic compound is disposed between two electrodes, wherein one or a plurality of organic layer contains the compound described in Formula I.

More specifically, the organic layer containing the compound described in Formula I further contains one or a plurality of a compound as described in Formula II or III:

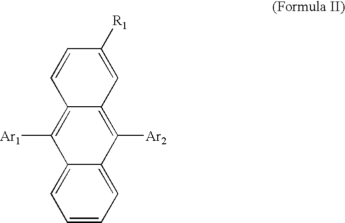

Wherein, Ar₁ and Ar₂ represents substituted or un-substituted aryl group from 6 to 20 carbon atoms while Ar₁ and Ar₂ may be of aryl groups identical with or different from each other; and R₁ represents direct chain or branch chain akyl group of hydrogen or from 1 to 4 carbon atoms.

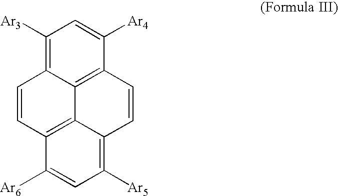

Where in Ar₃ through Ar₆ represents substituted or un-substituted aryl group from 6 to 20 carbon atoms; and Ar₃ through Ar₆ may be of aryl groups identical with or different from one another.

In the emission layer of the organic electro-luminescent device, when the novel blue organic compound of the present invention as expressed by Formula I is admixed as a secondary emission material into a primary emission material as that expressed in Formulae II and III, the emission efficiency and long service life of the device are improved to avail saturated color blue device by reducing generation of non-emission mechanism through a mechanism of direct incorporation once again in the secondary emission through energy transfer or electronics/electric holes between the primary emission material and the secondary emission material. In relation to the primary emission, a concentration of admixture of the secondary emission material falls between 0.01%˜50% by weight, and 0.5%˜20% by weight is preferred.

### BRIEF DESCRIPTION OF THE DRAWINGS

FIG. 1 is a ¹H-NMR spectrum of a compound (I)-1.

FIG. 2 is a ¹H-NMR spectrum of a compound (I)-10.

FIG. 3 is a sectional view of a summarized installation of an organic electro-luminescent device of the present invention.

### DETAILED DESCRIPTION OF THE PREFERRED EMBODIMENTS

A novel blue organic compound of the present invention as expressed in Formula I is essentially related to an organic material necessarily to be applied in an organic electro-luminescent and when applied as an emission material, a blue emission with high efficiency, saturated color and log device lifetime can be achieved.

Substituted and un-substituted aryl group of the novel blue organic compound of the present invention include but not limited to groups of phenyl, 2-tolyl, 3-tolyl, 4-tolyl, 1,3,5-trimethylbenzen, naphthyl, pyrene, phenanthryl, biphenyl, fluorine, and byphenylyl. Compounds listed below are examples better represent the novel blue organic compound of the present invention; and any possible derivative will be included in the teaching and scope of the present invention.

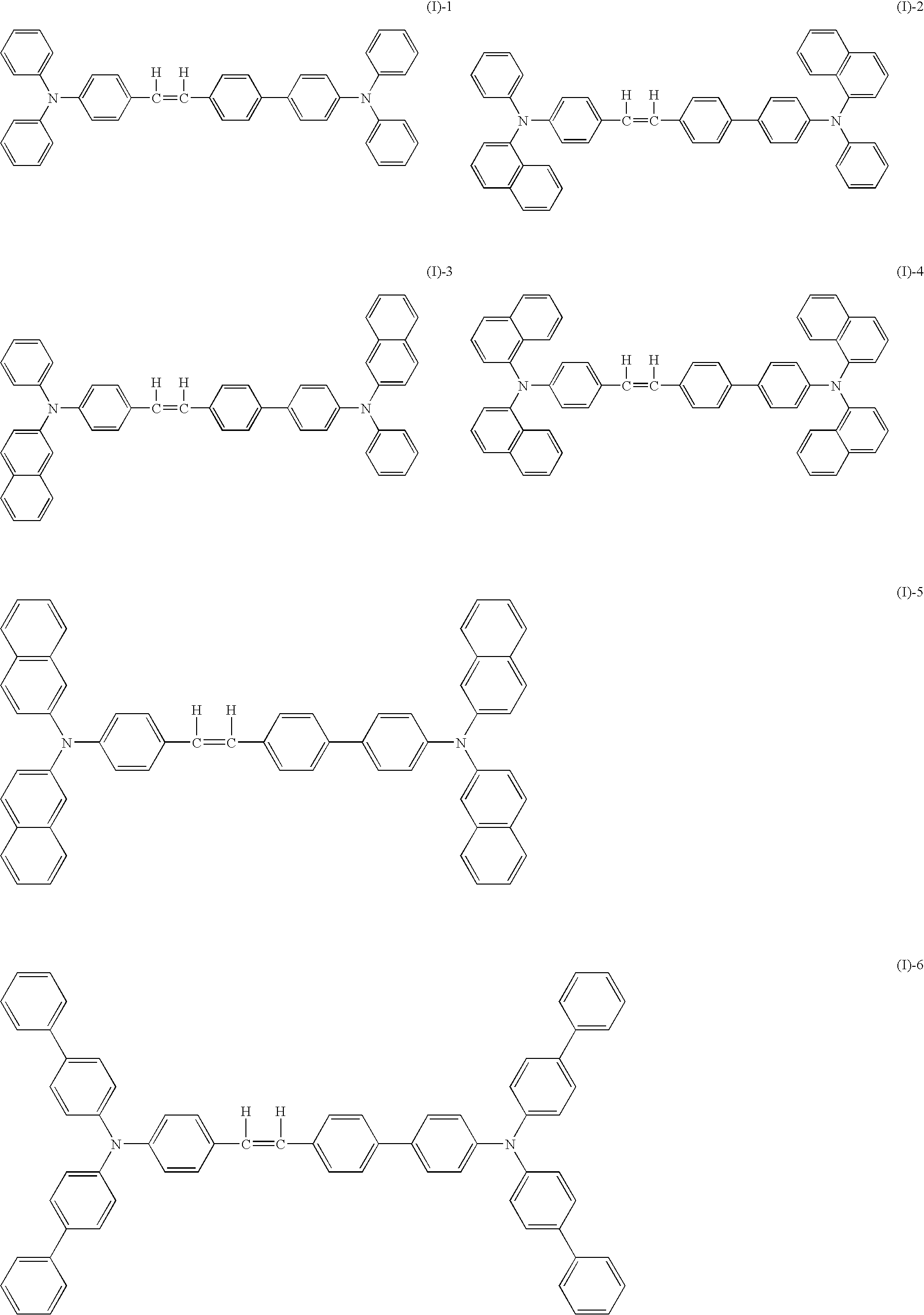

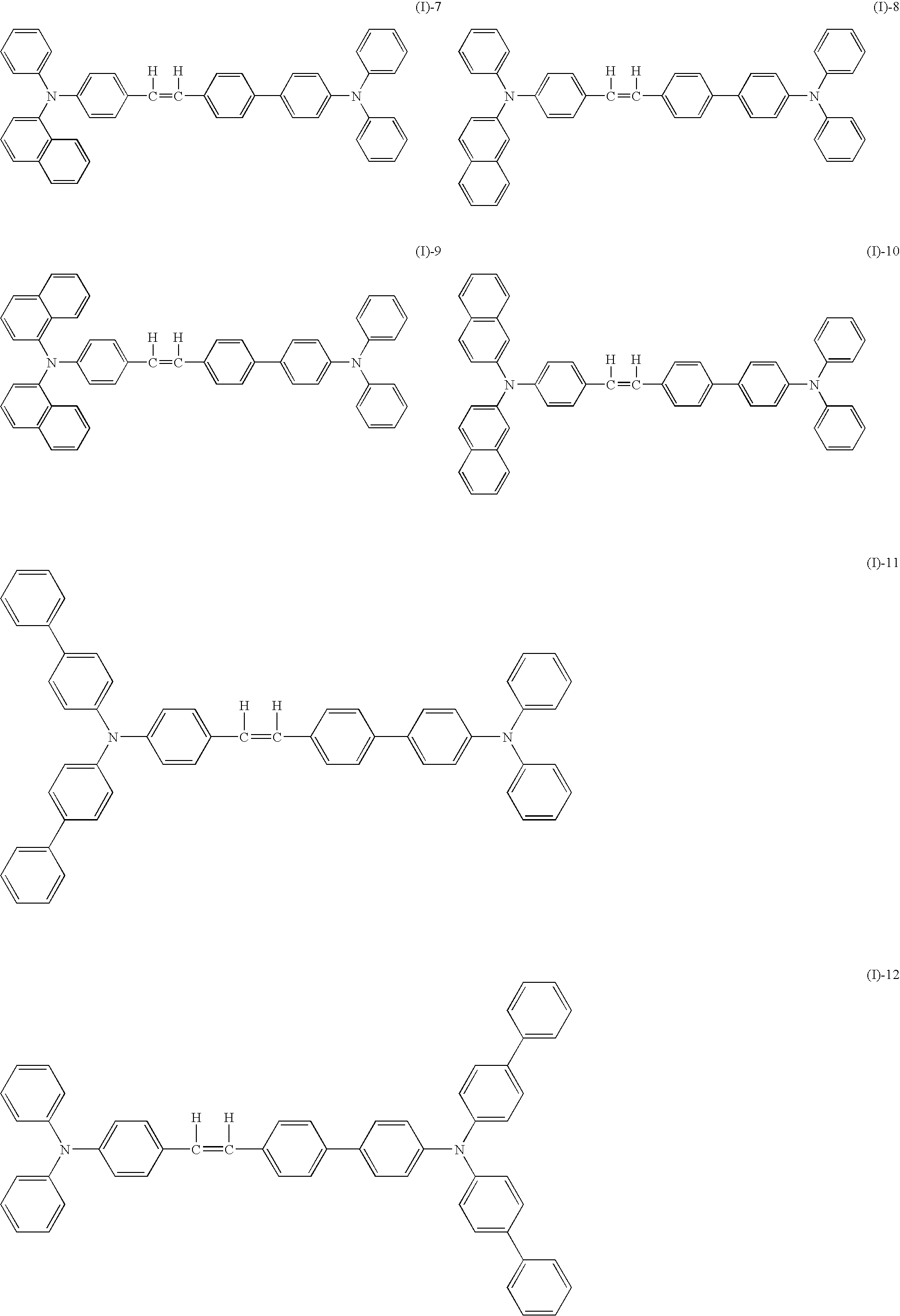

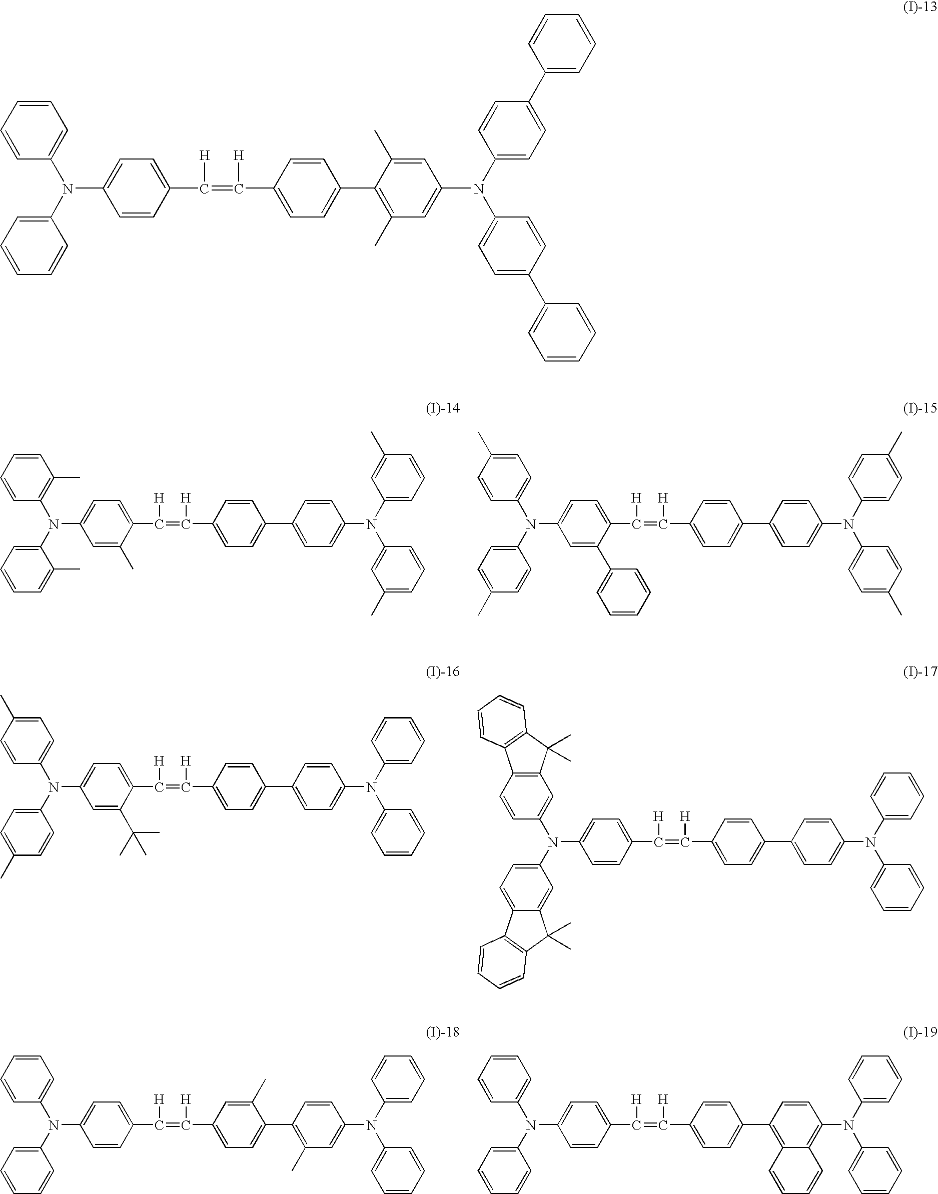

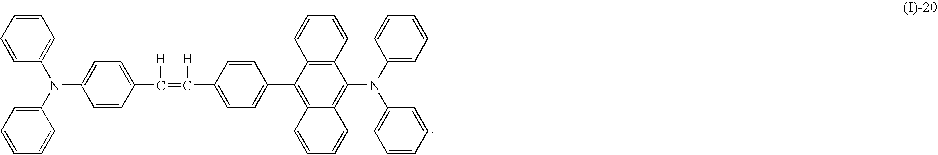

The organic electro-luminescent device is comprised of one or a plurality of pair of electrodes, a single layer or multiple layer structure containing an anode, a cathode, and organic compound is disposed between two electrodes, wherein one or a plurality of organic layer More specifically, the organic layer containing the compound described in Formula I further contains one or a plurality of a compound as described in Formula II or III:

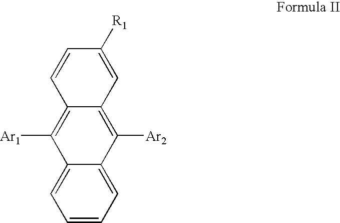

Wherein, Ar₁ and Ar₂ represents substituted or un-substituted aryl group from 6 to 20 carbon atoms while Ar₁ and Ar₂ may be of aryl groups identical with or different from each other; and R₁ represents direct chain or branch chain akyl group of hydrogen or from 1 to 4 carbon atoms.

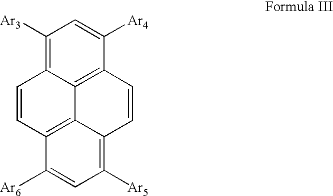

Wherein Ar₃ through Ar₆ represents substituted or un-substituted aryl group from 6 to 20 carbon atoms; and Ar₃ through Ar₆ may be of aryl groups identical with or different from one another.

In the emission layer of the organic electro-luminescent device, when the novel blue organic compound of the present invention as expressed by Formula I is admixed as a secondary emission material into a primary emission material as that expressed in Formulae II and III, the emission efficiency and long service life of the device are improved to avail saturated color blue device by reducing generation of non-emission mechanism through a mechanism of direct incorporation once again in the secondary emission through energy transfer or electronics/electric holes between the primary emission material and the secondary emission material. In relation to the primary emission, a concentration of admixture of the secondary emission material falls between 0.01%˜50% by weight, and 0.5%˜20% by weight is preferred.

Compounds listed below are examples better represent those expressed in Formulae II and II; and any possible derivative will be included in the teaching and scope of the present invention.

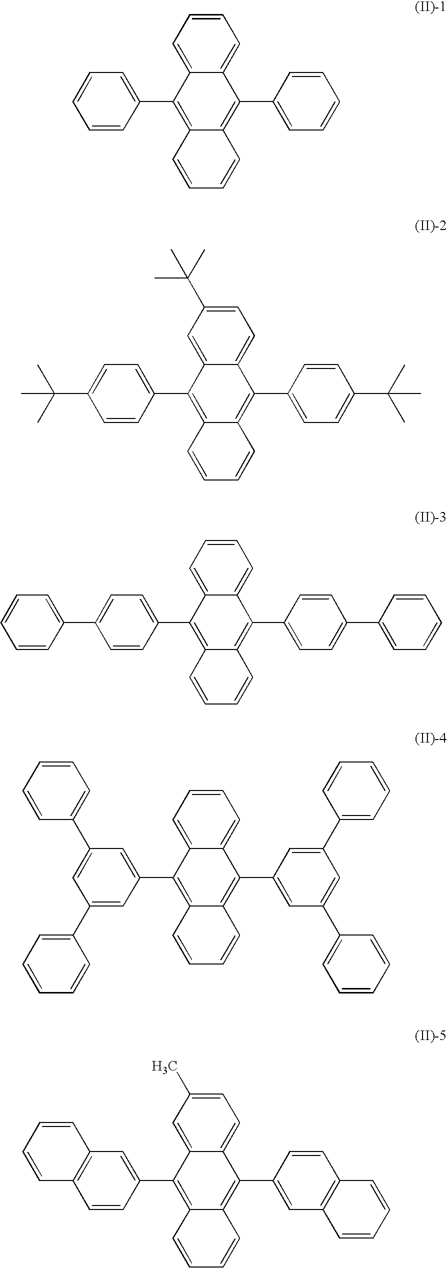

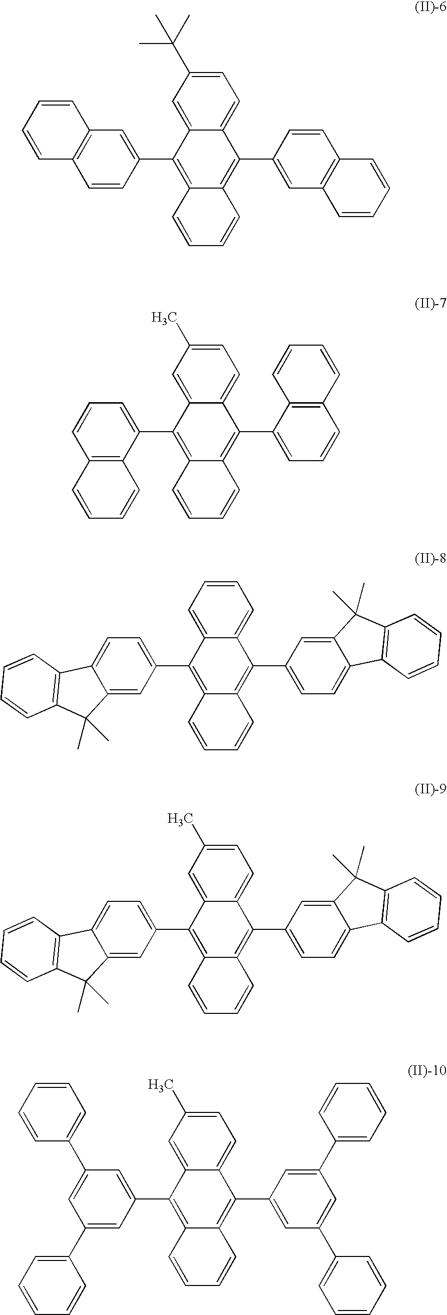

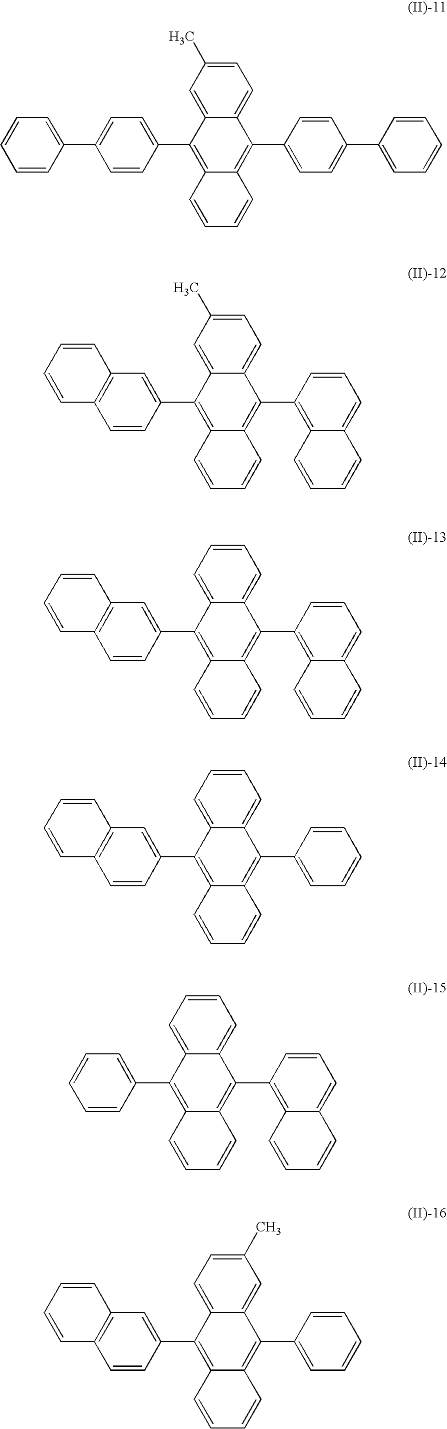

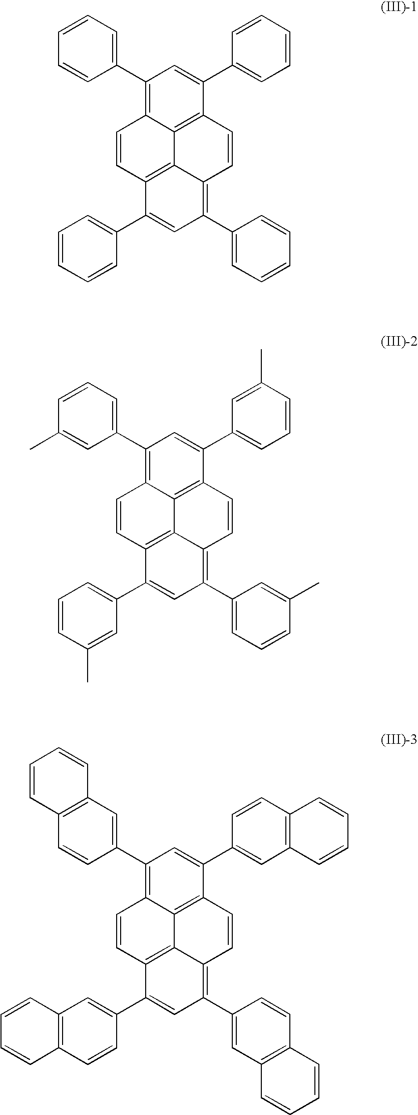

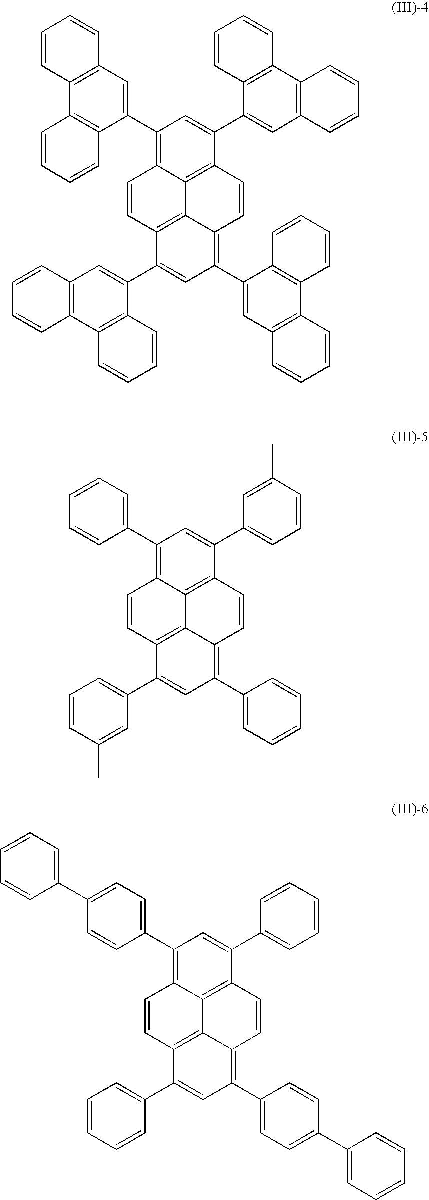

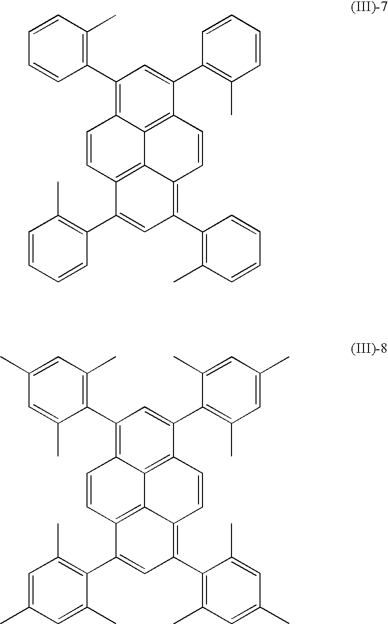

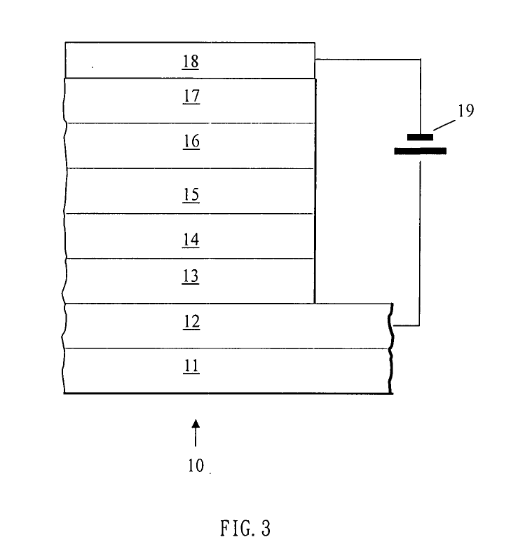

As illustrated in FIG. 3 for a summary sectional view of a preferred embodiment of the present invention, an organic electro-luminescent device (OLED) 10 is comprised of a transparent vitreous or plastic substrate 11 disposed thereon a transparent conduction anodic layer 12; an organic electric hole implantation material is then disposed on a surface of the anodic layer 12 to form an electric hole implantation 13. An organic electric hole transmission material is then disposed on a surface of the electric hole implantation layer 13 to form an organic electric hole transmission layer 14; an emission organic layer 15 made of a primary emission material admixed with a secondary emission material is disposed on a surface of the transmission layer 14; an electronic transmission layer 16 made of an electronic transmission material is disposed on a surface of the emission organic layer 15; an electronic implantation layer 17 made of an electronic implantation material is disposed on a surface of the electronic transmission layer 16; and finally, a metal conduction layer 18 is disposed on a surface of the electronic implantation layer 17 to form a cathode.

In this preferred embodiment, the conduction anodic layer 12 is of p-contact and the conduction cathode 18 is of n-contact. A negative terminal from a source 19 is connected to the conduction layer 18 and a positive terminal is connected to the conduction layer 12. When a potential is applied through the source 19 at where between the layer 12 and the layer 18, electronics implanted from n-contact (the layer 18) pass through the electronic implantation layer 17 and the organic electronic transmission layer 16 to enter into the organic emission layer 15; and electric holes implanted from p-contact (the layer 12) pass the organic electric hole implantation layer 13 and the organic electric hole layer 14 to enter into the organic emission layer 15. Electronics and electric holes are incorporated once again in the organic emission layer 15 to radiate photons.

In this preferred embodiment, the electronic implantation layer of the OLED 10 may be of LiF, 8-quinolinolato lithium (Liq), or 8-quinolinolato sodium (Naq); and the electronic transmission layer is made of any of the following materials:

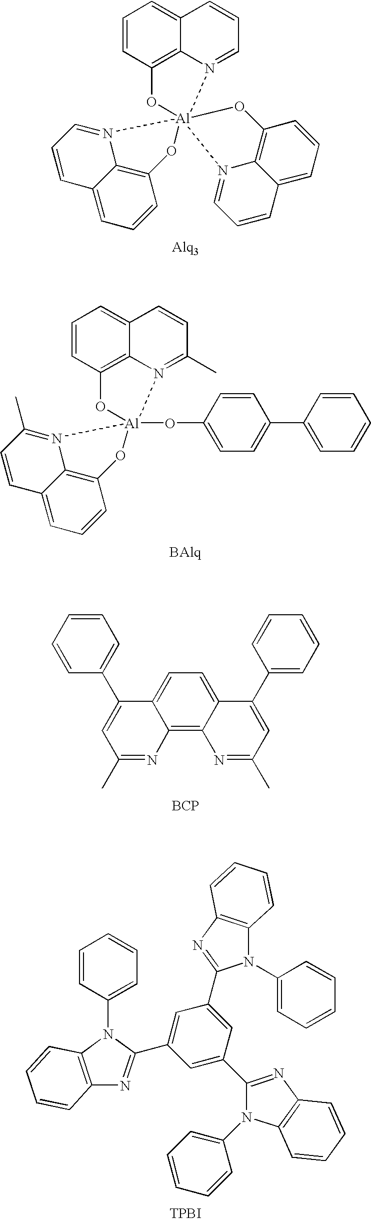

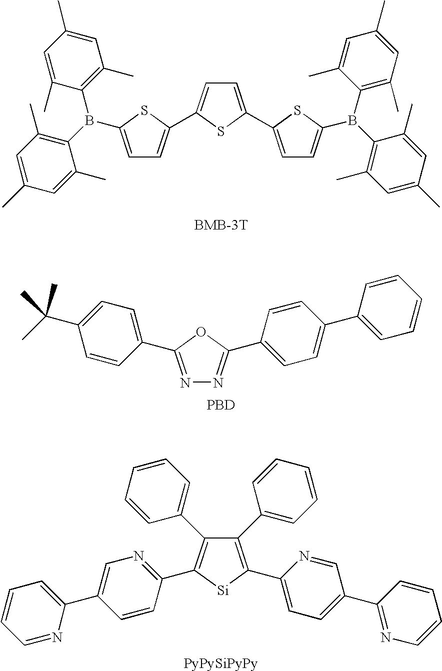

While the electric hole transmission layer is comprised of any of the following materials:

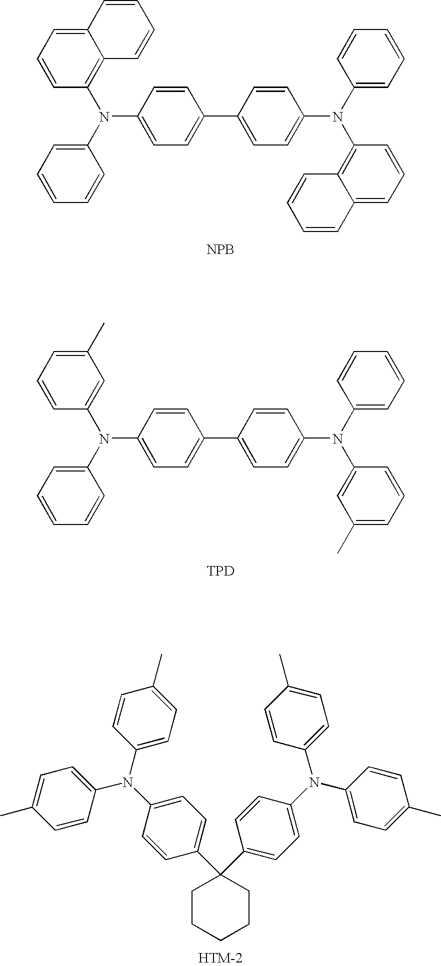

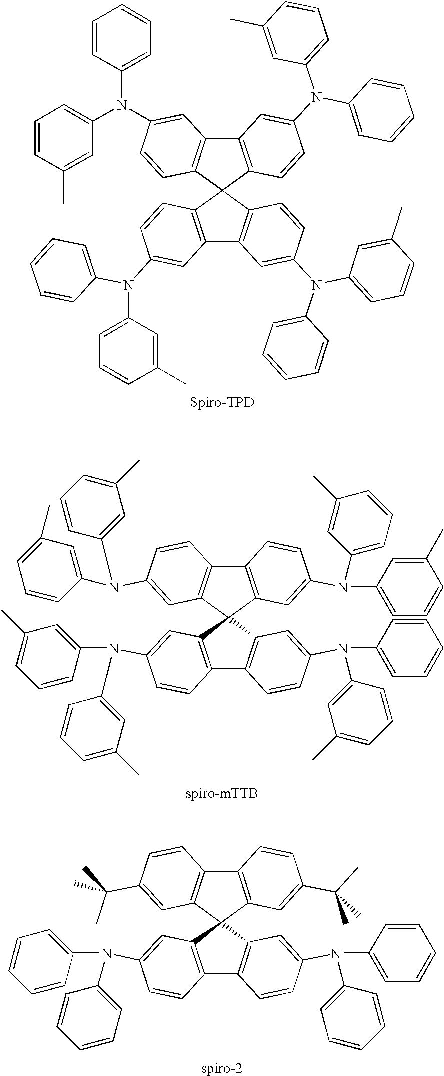

The electric hole implantation layer is comprised of any of the following materials: Fluorocarbon polymer, Poly(3,4-ethylene dioxythiophene)-Poly(styrenesulfonate), N,N′-diphenyl-N,N′-bis[N-phenyl-N-1-naphthyl (4-aminophenyl)]benzidine and their derivatives.

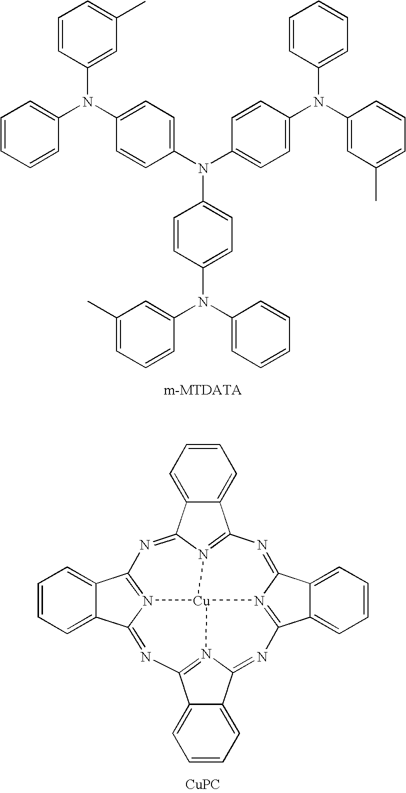

A first and a second preferred embodiments and detailed description of synthetic methods of the novel blue organic compound of the present invention in terms of synthetic route are given below; however, these preferred embodiments do not in any way limit the scope of the present invention.

### Preferred Embodiment 1: Synthetic Method and Route of Compound (I)-1

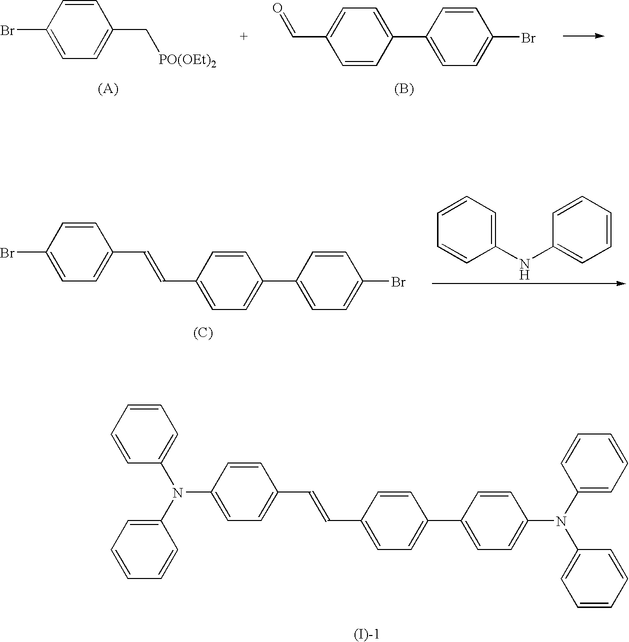

Synthetic Method of Medium (A):

Fetch a 100 ml triple-neck flask and add 25.8 g of 4-bromobenzyl bromide (0.1 mole) and 35 ml (0.2 mole) of triethyl phosphite and heat by circulation the flask at an temperature of 200° C. for 24 hours. Upon is achieving complete reaction, extra triethyl phosphite and product are fractionally distilled at a reduced pressure to avail 27.6 g of product at a production rate of 90%.

Synthetic Method of Medium (B):

Add into a 100 ml tri-neck flask 2 g (6.41 mmole) of 4,4′-dibromobiphenyl and 20 ml of dehydrated THF, and slowly drip 3.93 ml (6.41 mmole) of n-butyllithium solution (1.63M of hexane solution) in the presence of nitrogen at −78° C. Wait for thirty minutes upon completing the drip of n-butyllithium solution before slowing adding 20 ml of DMF to allow reaction temperature to gradually return to room temperature, followed with blending for two hours. Pour the solution in the flask into water and extracted using ethyl acetate and perform hexane and acetone chromatography for purification to avail 1.4 g of product at a production rate of 83%.

Synthetic Method of Medium (C):

Pour 20 ml of DMF in a 100 ml tri-neck flask and dissolve 1 g (3.3 mmole) of the medium (A) and 0.86 g (3.3 mmole) of the medium (B) in the flask, then add 0.48 g (5 mmole) of NaOtBu during ice bath and blend the solution at temperature for 24 hours. Once complete reaction is achieved, pour the reactants into water, filtrate the solids and bake them to dry to avail 1.1 g of product at a production rate of 80%.

Synthetic Method for Compound (I)-1

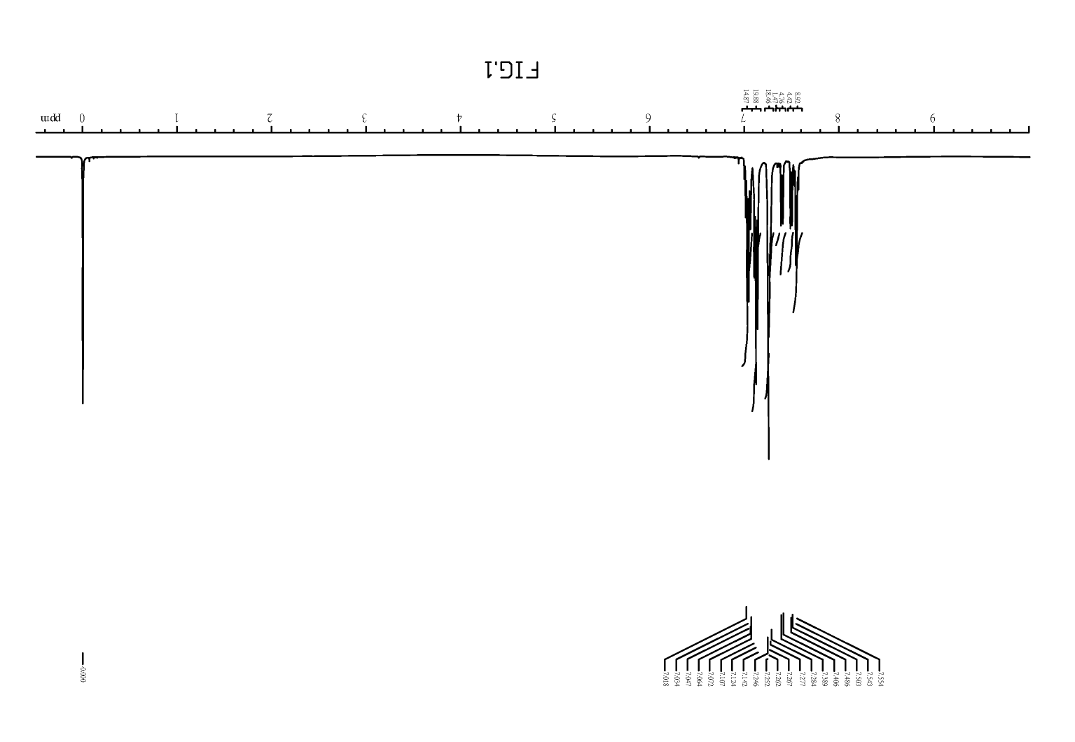

Add 1 g (2.4 mmole) of the medium (c), 0.9 g (5.3 mmole) of diphenylamine, 16 mg (0.07 mmole) of palladium(II)acetate, 28 ml (0.14 mmole) of tri(t-butyl)phosphine, and 0.7 g (7.2 mmole) of NaOtBu in a 100 ml tri-neck flask containing 50 ml of toluene to be heated in circulation for hours and cured until cooling down to room temperature before being extracted using ethyl acetate and water to take an organic layer. The layer is then concentrated and dried to be further purified by means of submilation with a final product to be confirmed through NMR, Mass and EA. FIG. 1 shows a NMR spectrum of the compound II)-1.

### Preferred Embodiment 2: Synthetic Method and Route of Compound (I)-10

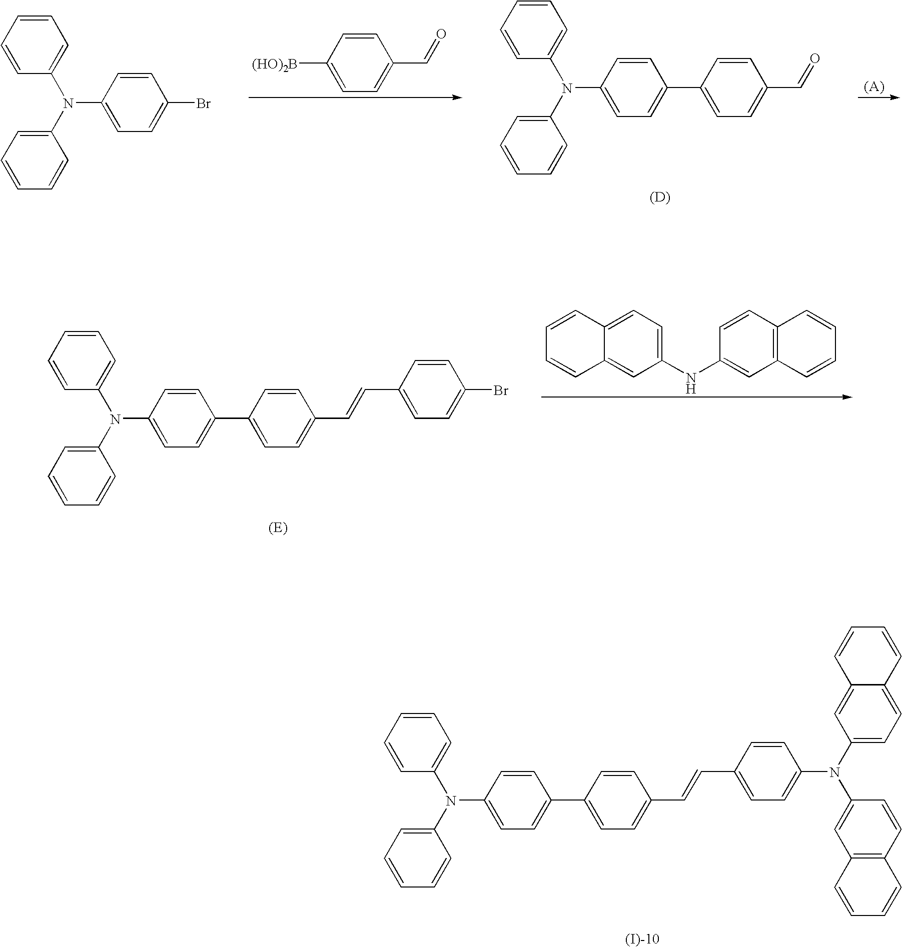

Synthetic Method of Medium (D):

Add into a 250 ml tri-neck flask 10 g (31 mmole) of 4-bromophenyl-diphenylamine, 100 ml of toluene, 10 ml of ethanol, 7.6 g (37.8 mmole) of 4-formylbenzeneboronic acid, 50 ml water solution of 2M soliem carbonate and 1.08 g (0.935 mmole) of tetrakis(triphenylphosphine)palladium(0) to be heated in circulation for 12 hours, extracted with ethyl acetate, added with proper amount of dehydrated magnesium sulfide, and concentrated to undergo ethanol bath for brown solids to avail 9.6 g of yellow solids at a production rate of 89%.

Synthetic Method of medium (E):

Dissolve 6 g (17.2 mmole) of the medium (D), 4.8 g (15.6 mmole) of the medium (A), and 2.7 g (24.1 mmole) of KOtBu in a 150 ml tri-neck flask containing 80 ml of DMF, and blend the solution for 24 hours at room temperature; upon the reaction is completed, pour reactants into a solution of methyl alcohol and water mixed at the ratio of 1:1 by volume and filtrate for solids to be dried for availing 14.5 g of rough product; the product is then crystallized using ethyl acetate and hexane for purification to avail 6.3 g of transparent crystal at a production rate of 80%.

Synthetic Method of Compound (I)-10:

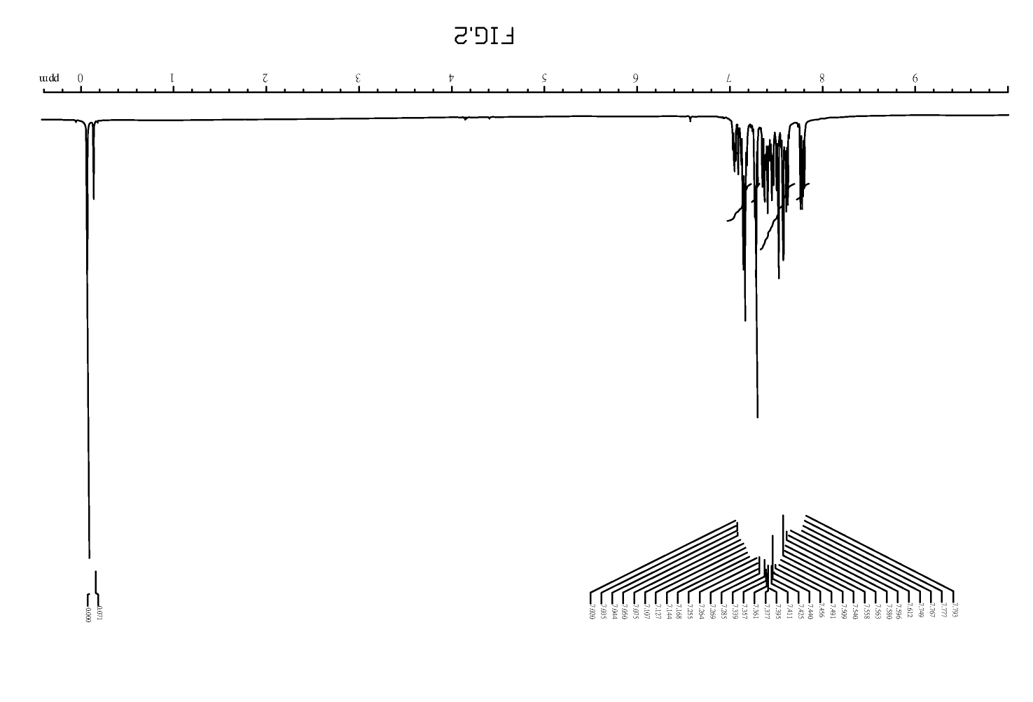

Add 1 g (2 mmole) of the medium (E), 0.6 g (2.2 mmole) of di-2-naphthylamine, 14 mg (0.06 mmole) of palladium(II)acetate, 24 mg (0.12 mmole) of tri(t-butyl)phosphine, and 0.3 g (3mmole) of NaOtBu in a 100 ml tri-neck flask containing 50 ml of toluene to be heated in circulation for 8 hours, then cured to room temperature, extracted with ethyl acetate and water for an organic layer; the organic layer is contractrated and dried to undergo further purification by sublimation with a final product to be confirmed using NMR, Mass and EA. FIG. 2 shows an NMR spectrum of the compound (I)-10.

Referring to the sectional view given in FIG. 3, a third, fourth, and fifth preferred embodiments and a first reference for comparison of the organic electro-luminescent device of the present invention are processed as follows:

### Preferred Embodiment 3—Production and Measurement of a Compound (I)-1 Based Device

(a) Rinse and oven dry an ITO glass with detergent and organic solution, then have a surface of the ITO glass plasma processed before being conducted with CHF₃ gas to treat the surface of the ITO with a plasma processor, a resultant CFx film functions as the electric hole implantation; and finally, the substrate is given organic film vapor disposition in a highly vacuumed environment.

(b) Have the electric hole transmission layer of (4,4′-bis[N-(1-naphthyl)-N-phenyl-amino]-biphenyl (NPB) vapor disposed in a thickness of 500 Å on the CFₓ coated ITO surface.

(c) Have both of the primary emission (II)-5 and the compound (I)-1 vapor disposed on the NPB layer to form a 400 Å emission layer, wherein, a ratio of the compound (I)-1 to the compound by volume is 7%.

(d) Have the electronic transmission layer, tris(8-quinolinol)aluminum (Alq₃) vapor disposed in a thickness of 100 Å on the emission layer.

(e) Have the electronic implantation,

Lithium fluoride (LiF) vapor disposed in a thickness of 10 Å on the electronic transmission layer.

Have aluminum vapor disposed on the electronic implantation to form a 2000 Å cathode.

The final product of the device is then conducted and measured with a light color meter for its luminance and luminance efficiency.

Under a drive amperage of 20 mA/cm², the EL device demonstrates its characteristics as shown in the table below:

| | |
|---|---|
| Drive voltage (volts) | 7.0 |
| Emission Luminance (cd/m²) | 872.3 |
| Emission Efficiency (cd/A) | 4.4 |
| CIE Coordinates, x | 0.15 |
| CIE Coordinates, y | 0.15 |
| Maximal emission peak (nm) | 452 |
| ½ Wave width (nm) | 64 |

### Preferred Embodiment 4: Production and Measurement of a Compound (I)-1 Based Device

Similar to the step described for the third preferred embodiment, wherein CFx in the electric hole implantation is replaced with 600 Å N,N′-diphenyl-N,N′-bis[N-phenyl-N-1-naphthyl(4-aminophenyl)]benzidine, the electric hole transmission layer is related to a 200 Å NPB, the emission layer is related to 300 Å 5% compound (I)-1 admixed in the compound (II)-7, the electronic transmission layer is related to 100 Å Alq3, and the electronic implantation is related to 10 Å LiF.

Under a drive amperage of 20 mA/cm², the EL device demonstrates its characteristics as shown in the table below:

| | |
|---|---|
| Drive voltage (volts) | 6.3 |
| Emission Luminance (cd/m²) | 1044.7 |
| Emission Efficiency (cd/A) | 5.2 |
| CIE Coordinates, x | 0.15 |
| CIE Coordinates, y | 0.13 |
| Maximal emission peak (nm) | 448 |
| ½ Wave width (nm) | 60 |

### Preferred Embodiment 5: Production and Measurement of a Compound (I)-10 Based Device

Same as that for the fourth preferred embodiment with the exception that the emission layer is related to 5% compound (I)-10 admixed in the compound (II)-7.

Under a drive amperage of 20 mA/cm², the EL device demonstrates its characteristics as shown in the table below:

| | |
|---|---|
| Drive voltage (volts) | 5.6 |
| Emission Luminance (cd/m²) | 1021.2 |
| Emission Efficiency (cd/A) | 5.1 |
| CIE Coordinates, x | 0.14 |
| CIE Coordinates, y | 0.14 |
| Maximal emission peak (nm) | 452 |
| ½ Wave width (nm) | 56 |

Reference 1 for Comparison: Production and Measurement of Compound DPAS-Based Device

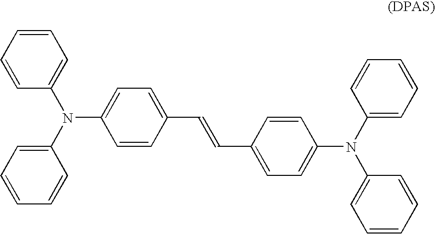

Same as that for the third preferred embodiment, wherein the ratio of the compound DPAS to the compound (II)-5 is 7% by volume.

Under a drive amperage of 20 mA/cm² the EL device demonstrates its characteristics as shown in the table below:

| | |
|---|---|
| Drive voltage (volts) | 6.7 |
| Emission Luminance (cd/m²) | 977.7 |
| Emission Efficiency (cd/A) | 4.9 |
| CIE Coordinates, x | 0.16 |
| CIE Coordinates, y | 0.21 |
| Maximal emission peak (nm) | 472 |
| ½ Wave width (nm) | 84 |

As told from those preferred embodiments and the reference for comparison, it appears that by admixing the novel blue organic compound of the present invention in a proper primary emission material, the organic electro-luminescent device achieves a blue emission with high efficiency, saturated color and long device lifetime. It is to be noted and any possible derivative will be included in the teaching and scope of the present invention

I claim:

1. A blue organic compound as expressed in Formula I below: 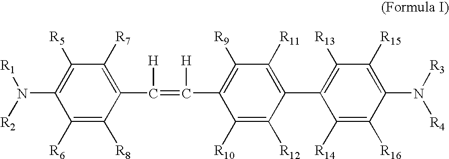
wherein R₁, R₂, R₃, and R₄ represent a substituted or unsubstituted aryl group from 6 to 20 carbon atoms, in which R₁, R₂, R₃, and R₄ may be identical with or different from each other, or R₁-R₂ and R₃-R₄ may be bridged to 5 to 7-membered carbocyclic ring; R5 to R16 represent hydrogen or a substituted or un-substituted alkyl or aryl group from 1 to 10 carbon atoms.

2. An organic electro-luminescent device comprising one or a plurality of pair of electrodes, a single layer or multiple layer structure containing an anode, a cathode, and organic compound is disposed between two electrodes, wherein one or a plurality of organic layer More specifically, the organic layer containing the compound described in Formula I further contains one or a plurality of a compound as described in Formula I: 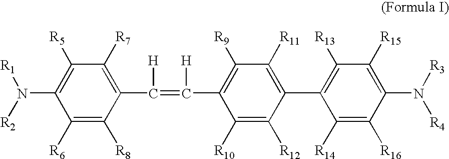
wherein R₁, R₂, R₃, and R₄ represent a substituted or unsubstituted aryl group from 6 to 20 carbon atoms, in which R₁, R₂, R₃, and R₄ may be identical with or different from each other, or R₁-R₂ and R₃-R₄ may be bridged to 5 to 7-membered carbocyclic ring; R5 to R16 represent hydrogen or a substituted or un-substituted alkyl or aryl group from 1 to 10 carbon atoms.

3. The organic electro-luminescent device as claimed in claim 2, the organic layer containing the compound expressed in Formula I further contain a compound as expressed in Formula II below: 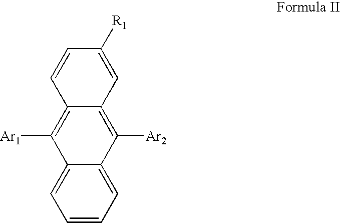
wherein, Ar₁ and Ar₂ represents substituted or un-substituted aryl group from 6 to 20 carbon atoms while Ar₁ and Ar₂ may be of aryl groups identical with or different from each other; and R₁ represents direct chain or branch chain akyl group of hydrogen or from 1 to 4 carbon atoms.

4. The organic electro-luminescent device as claimed in claim 2, the organic layer containing to the compound expressed in Formula I further contain a compound as expressed in Formula III below: 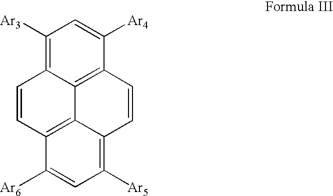
wherein Ar₃ through Ar₆ represents substituted or un-substituted aryl group from 6 to 20 carbon atoms; and Ar₃ through Ar₆ may be of aryl groups identical with or different from one another.

5. The organic electro-luminescent device as claimed in claim 2, wherein the organic layer containing the compound as expressed by Formula I functions as an emission layer.

6. The organic electro-luminescent device as claimed in claim 3, wherein the organic layer containing the compounds as respectively expressed by Formulae I and II functions as an emission layer.

7. The organic electro-luminescent device as claimed in claim 4, wherein the organic layer containing the compounds as respectively expressed by Formulae I and III functions as an emission layer.

8. The organic electro-luminescent device as claimed in claim 6, wherein an admixing ratio of the compound expressed in Formula I to the compound expressed in Formula II ranges from 0.5% to 20% by weight.

9. The organic electro-luminescent device as claimed in claim 7, wherein an admixing ratio of the compound expressed in Formula I to the compound expressed in Formula III ranges from 0.5% to 20% by weight.

10. An organic electro-luminescent device including
a substrate;
a conduction anodic layer disposed on the substrate;
an organic electric hole implantation disposed on the conduction anodic layer;
an organic electric hole transmission layer disposed on the organic electric hole implantation;
a light emission organic layer disposed on the organic electric hole transmission layer;
an electronic transmission layer disposed on the light emission organic layer,
an electronic implantation disposed on the electronic transmission layer; and
a metal conduction layer disposed on the electronic implantation to form a cathode, wherein the light emission layer further includes a compound as expressed by Formula I: 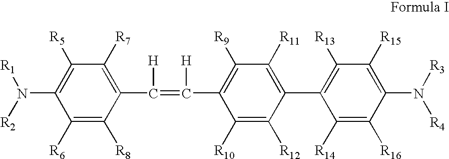
wherein R₁, R₂, R₃, and R₄ represent a substituted or unsubstituted aryl group from 6 to 20 carbon atoms, in which R₁, R₂, R₃, and R₄ may be identical with or different from each other, or R₁-R₂ and R₃-R₄ may be bridged to 5 to 7-membered carbocyclic ring. R5 to R16 represent hydrogen or a substituted or unsubstituted alkyl or aryl group from 1 to 10 carbon atoms

11. The organic electro-luminescent device as claimed in claim 10, wherein the substrate relates to a vitreous or plastic material.

12. The organic electro-luminescent device as claimed in claim 10, wherein the emission organic layer further includes a compound as expressed in Formula II below: 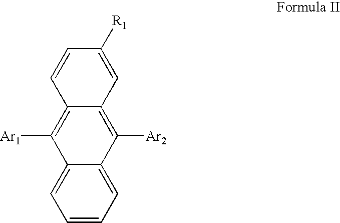
wherein, Ar₁ and Ar₂ represents substituted or un-substituted aryl group from 6 to 20 carbon atoms while Ar₁ and Ar₂ may be of aryl groups identical with or different from each other; and R₁ represents direct chain or branch chain akyl group of hydrogen or from 1 to 4 carbon atoms.

13. The organic electro-luminescent device as claimed in claim 10, wherein the emission organic layer further includes a compound as expressed in Formula III below: 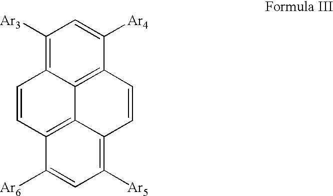
wherein Ar₃ through Ar₆ represents substituted or un-substituted aryl group from 6 to 20 carbon atoms; and Ar₃ through Ar₆ may be of aryl groups identical with or different from one another.

14. The organic electro-luminescent device as claimed in claim 12, wherein an admixing ratio of the compound expressed in Formula I to the compound expressed in Formula II ranges from 0.5% to 20% by weight.

15. The organic electro-luminescent device as claimed in claim 13, wherein an admixing ratio of the compound expressed in Formula I to the compound expressed in Formula III ranges from 0.5% to 20% by weight.

16. The organic electro-luminescent device as claimed in claim 10, wherein the materials for the electronic implantation is selected form LiF, 8-quinolinolato lithium (Liq), 8-quinolinolato sodium (Naq) or any of their combinations.

17. The organic electro-luminescent device as claimed in claim 10, wherein the materials for the electronic transmission layer is selected from Alq₃, BAlq, BCP, TPBI, BMB-3T, PBD, PyPySiPyPy or any of their combinations.

18. The organic electro-luminescent device as claimed in claim 10, wherein the material for the electric hole transmission layer is selected from NPB, TPD, HTM-2,Spiro-TPD, spiro-mTTB, spiro-2 or any of their combinations.

19. The organic electro-luminescent device as claimed in claim 10, wherein the material for the electric hole implantation is selected from CFx, Poly(3,4-ethylene dioxythiophene)-Poly(styrenesulfonate),N,N′-diphenyl-N,N′-bis[N-phenyl-N-1-naphthyl(4-aminophenyl)]benzidine and their derivative, m-MTDATA, CuPc or any of their combinations.

20. The novel blue organic compound as claimed in claim 1, wherein R₁-R₅, R₂-R₆, R₃-R₁₅, R₄-R₁₆, R₅-R₇, R₆-R₈, R₉-R₁₁, R₁₀-R₁₂, R₁₃-R₁₅ and R₁₄-R₁₆ may be bridged to a saturated or unsaturated carbocyclic ring from 3 to 10 carbon atoms.

21. The organic electro-luminescent device as claimed in claim 2, wherein R₁-R₅, R₂-R₆, R₃-R₁₅, R₄-R₁₆, R₅-R₇, R₆-R₈, R₉-R₁₁, R₁₀-R₁₂, R₁₃-R₁₅ and R₁₄-R₁₆ may be bridged to a saturated or unsaturated carbocyclic ring from 3 to 10 carbon atoms.

22. The organic electro-luminescent device as claimed in claim 10, wherein, R₁-R₅, R₂-R₆, R₃-R₁₅, R₄-R₁₆, R₅-R₇, R₆-R₈, R₉-R₁₁, R₁₀-R₁₂, R₁₃-R₁₅ and R₁₄-R₁₆ may be bridged to a saturated or unsaturated carbocyclic ring from 3 to 10 carbon atoms.
